(19) United States
(12) Patent Application Publication
(10) Pub. No.: US 2009/0311318 A1
(43) Pub. Date: Dec. 17, 2009

(54) SUBSTITUTED (S)-BENZOXAZINONES

(75) Inventors: Daniel Holsworth, San Diego, CA (US); William Park, Westerly, RI (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: P&H Therapeutics, Inc., Providence, RI (US)

(21) Appl. No.: 12/484,119

(22) Filed: Jun. 12, 2009

Related U.S. Application Data
(60) Provisional application No. 61/061,562, filed on Jun. 13, 2008.

(51) Int. Cl. A61K 31/538; C07D 413/10; A61K 9/22; A61K 9/52

(52) U.S. Cl. 424/457; 544/105; 514/230.5; 424/468

(57) ABSTRACT

The present invention provides enantiomerically pure substituted (S)-benzoxazinone compounds and an extended release formulation of the compounds. The compounds exhibit potent renin inhibition activities and improved bioavailability.

### CROSS-REFERENCES TO RELATED APPLICATIONS

This application claims priority to U.S. Provisional Patent Application No. 61/061,562 filed Jun. 13, 2008, which application is incorporated herein by reference in its entirety and for all purposes.

### STATEMENT AS TO RIGHTS TO INVENTIONS MADE UNDER FEDERALLY SPONSORED RESEARCH AND DEVELOPMENT

NOT APPLICABLE

### BACKGROUND OF THE INVENTION

Hypertension is a leading risk factor for cardiovascular disease, such as congestive heart failure, stroke, and myocardial infarction. Renin is an endopeptidase (molecular weight about 40,000) produced and secreted by the juxtaglomerular cells of the kidney, which cleaves the naturally-occurring plasma glycoprotein and antiotensinogen. Renin cleaves angiotensinogen, its protein substrate, to split off the hemodynamically-inactive N-terminal decapeptide, angiotensin I, which is converted in the lungs, kidney or other tissue by angiotensin-converting enzyme to the potent pressor octapeptide, angiotensin II. Angiotensin II is known to be a potent pressor substance, i.e., a substance that is capable of inducing a significant increase in blood pressure, and is believed to act by causing the constriction of blood vessels and the release of the sodium-retaining hormone aldosterone from the adrenal gland. Thus, the renin-angiotensinogen system has been implicated as a causative factor in certain forms of hypertension and congestive heart failure (Stanton, Journal of the Renin-Angiotensin-Aldosterone System 2003, 4, 6; and Rosenberg, et al. Antihypertensive Drugs 1997, 77).

Inhibitors of angiotensin I converting enzyme have proven useful in the modulation of the renin-angiotensin system. Consequently, specific inhibitors of the limiting enzymatic step that ultimately regulates angiotensin II production, the action of renin on its substrate, are sought as effective therapeutic agents in the treatment of hypertension, and congestive heart failure. However, attempts to inhibit renin have been centered on using high molecular weight transition state mimetics based on the angiotensinogen backbone. These peptidomimetic inhibitors suffered from poor PK properties such as low oral bioavailability, short duration of action, low potency, and/or cost of synthesis. Compared to the racemic analogs, enantiomerically pure small molecule compounds have been shown to exhibit different pharmacokinetic profiles and activities (see, Powell, et al. Bioorganic & Medicinal Chemistry 2007, 15, 5912-49).

Therefore, there is a need to develop enantiomerically pure small molecule renin inhibitors that have an improved potency, improved bioavailability, improved safety, stability and an optimized renin inhibitory activity. The present invention meets these and other needs.

### BRIEF SUMMARY OF THE INVENTION

In one aspect, the present invention provides a compound having formula (I):

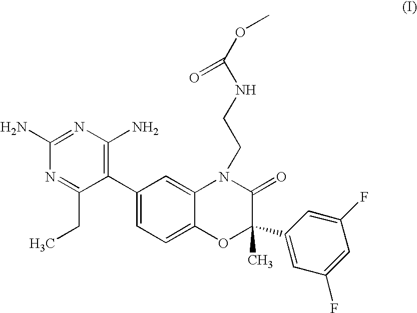

and a pharmaceutically acceptable salt or solvate thereof, wherein the stereochemical configuration at the carbon atom bearing —CH₃ and 3,5-difluorophenyl is (S).

In another aspect, the present invention provides a pharmaceutical composition. The composition includes a pharmaceutically acceptable excipient or carrier and a compound of formula (I).

In yet another aspect, the present invention provides methods for preparing a compound of formula (I) and key intermediates as set forth in synthetic Schemes 1-3 (FIGS. 1-3).

In still another aspect, the present invention provides an extended release formulation. The formulation includes a core comprising a compound of formula (I) or a pharmaceutically acceptable salt thereof, a pharmaceutically acceptable excipient and an extended release component, and an extended release coating associated with the core to provide for sustained release of compound of formula (I).

### BRIEF DESCRIPTION OF THE DRAWINGS

FIG. 1 illustrates one synthetic approach to substituted (S)-benzoxazinones according to an embodiment the present invention.

FIG. 2 illustrates another synthetic approach to substituted (S)-benzoxazinones according to an embodiment the present invention.

FIG. 3 illustrates a synthetic approach to substituted (S)-benzoxazinones according to an embodiment the present invention.

FIGS. 4A-4C illustrate the mean blood pressure (BP) profile of SHR rats after administration of compound (S)-10. A: mean BP average. B: systolic BP mean. C: Diastolic BP mean.

FIG. 5 illustrates the heart rate mean of SHR rats after administration of compound (S)-10.

FIGS. 6A-6C illustrate the BP change of SHR rats from baseline after administration of compound (S)-10. A: mean BP change from baseline. B: systolic BP mean change from baseline. C: diastolic BP mean change from baseline.

FIG. 7 illustrates heart rate mean change of SHR rats from baseline after administration of compound (S)-10.

### DETAILED DESCRIPTION OF THE INVENTION

### I. DEFINITIONS

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occuring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and deleterious to the recipient thereof.

The term “pharmaceutically acceptable excipient or carrier” means one or more excipients that are useful in preparing a pharmaceutical composition. Excipients are generally safe, non-toxic and neither biologically nor otherwise undesirable, and include excipients that are acceptable for veterinary use as well as human pharmaceutical use. “A pharmaceutically acceptable carrier” as used in the specification and claims includes both one and more than one such carrier.

Certain compounds of the present invention can exist in unsolvated forms as well as solvated forms, including hydrated forms. “Hydrate” refers to a complex formed by combination of water molecules with molecules or ions of the solute. “Solvate” refers to a complex formed by combination of solvent molecules with molecules or ions of the solute. The solvent can be an organic compound, an inorganic compound, or a mixture of both. Solvate is meant to include hydrate. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “therapeutically effective amount” refers to the amount of a compound that, when administered to a mammal for preventing or treating a disease, is sufficient to effect such prevention or treatment for the disease. The “therapeutically effective amount” will vary depending on the compound, the disease and its severity and the age, weight, etc., of the mammal to be treated.

As used herein, “administering” means oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “subject” means mammals, including, without limitation, humans, domestic animals (e.g., dogs or cats), farm animals (cows, horses, or pigs), goats, monkeys, rabbits, mice, and laboratory animals.

A “dosage form” or “dosage formulation” means a unit of administration of an active agent. Examples of dosage formulations include tablets, capsules, injections, suspensions, liquids, emulsions, creams, ointments, suppositories, inhalable formulations, transdermal formulations, and the like.

The term “extended release” or “sustained release” is defined herein as release of a pharmaceutical agent in a continuous manner over a prolonged period of time.

By “controlled-release” is meant a dosage form in which the release of the active agent is controlled or modified over a period of time. Controlled can mean, for example, extended-, sustained-, delayed- or pulsed-release at a particular time. Alternatively, controlled can mean that the release of the active agent is extended for longer than it would be in an immediate-release dosage form, e.g., at least over several hours.

By “prolonged period of time” it is meant a continuous period of time of greater than about 1 hour, preferably, greater than about 4 hours, more preferably, greater than about 8 hours, more preferably greater than about 12 hours, more preferably still, greater than about 16 hours up to more than about 24 hours.

By “immediate-release” is meant a conventional or non-modified release in which greater then or equal to about 75% of the active agent is released within two hours of administration, specifically within one hour of administration.

As used herein, the term “delayed release” refers to a pharmaceutical preparation that passes through the stomach intact and dissolves in the small intestine.

“Dosing under fasting conditions” is defined as when the dosage is administered orally with 240 ml of room temperature water after subjects are fasted overnight for at least 10 hours. No fluid, except that given with drug administration, will be allowed from 1 hour prior to dose administration until 1 hour after dosing. At 2 hours post-dose, subjects may consume 240 ml of room temperature water.

### II. GENERAL

The present invention relates to optically active and enantiomerically pure (S)-substituted benzoxazinones that are aspartic acid protease inhibitors in general and renin inhibitors in particular. Surprisingly, the (S)-enantiomers of the compounds have improved potencies, improved and enhanced bioavailability, improved safety, optimized renin inhibitory activities, and enhanced metabolic stabilities.

### III. COMPOUNDS

In one aspect, the present invention provides a compound having formula (I):

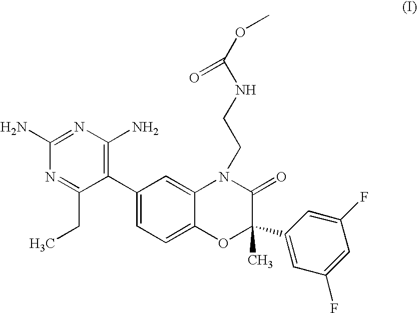

and pharmaceutically acceptable salts or solvates thereof, wherein the stereochemical configuration at the carbon atom bearing —CH₃ and 3,5-difluorophenyl is (S).

### Preparation of Compounds

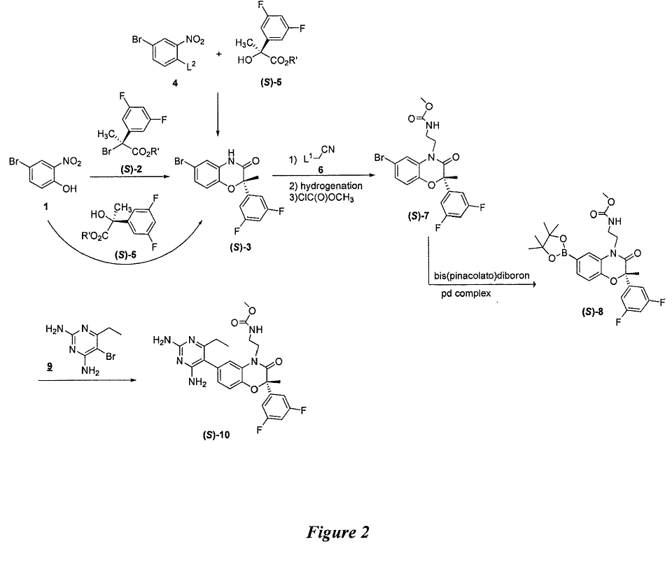

Compounds of the present invention can be prepared using readily available starting materials or known intermediates. Examples of starting materials available from commercial suppliers include, but are not limited to, 4-bromo-2-nitrophenol, 4-bromo-1-fluoro-2-nitrobenzene, 5-bromo-3-nitropyridine-2-ol, 2-hydroxy carboxylic esters, 2-bromo carboxylic esters, 2-substituted racemic 2-hydroxy carboxylic esters, 2-substituted optically active 2-hydroxy carboxylic esters, 2-substituted racemic 2-bromo carboxylic esters and 2-substituted optically active 2-bromo carboxylic esters, e.g., compound (S)-2 (see, FIG. 2). Other starting materials can be prepared according to the literature procedures. The 2-hydroxy or 2-bromo carboxylic esters include those where the 2-position is further substituted with one or two non-hydrogen substituents. The 2-hydroxy or 2-bromo carboxylic esters can be either optically active or racemic. Non commercially available 2-hydroxy or 2-bromo carboxylic esters can be synthesized by alkylation or arylation of 2-hydroxy or 2-bromo carboxylic esters, where the hydroxyl groups can be either protected or unprotected. For alkylation, the reaction can be carried out in the presence of a base, such as lithium diisopropylamide (LDA) (see, Williams, et al. J. Org. Chem. 1980, 45, 5082; and Rathke, et al. J. Am. Chem. 1971, 93, 2320). For arylation, the reaction can be carried out in the presence of a copper halide or a palladium complex (see, Lindley, et al. Tetrahedron 1984, 40, 1433-1456; Uno, et al. Synthesis 1985, 506; Hartwig, et al. J. Am. Chem. Soc., 2002, 124, 12557-12565; and Marion, et al. J. Org. Chem., 2006, 71, 3816-3821). The choice of appropriate reaction conditions is within the ability of those of skill in the art.

Optically active 2-hydroxy or 2-bromo carboxylic esters can be readily prepared by using chiral auxillaries or asymmetric catalysts (see, Hartwig, et al. J. Am. Chem. Soc. 2004, 126, 5182-5191). The choice of appropriate reaction conditions can be readily established by those of skill in the art.

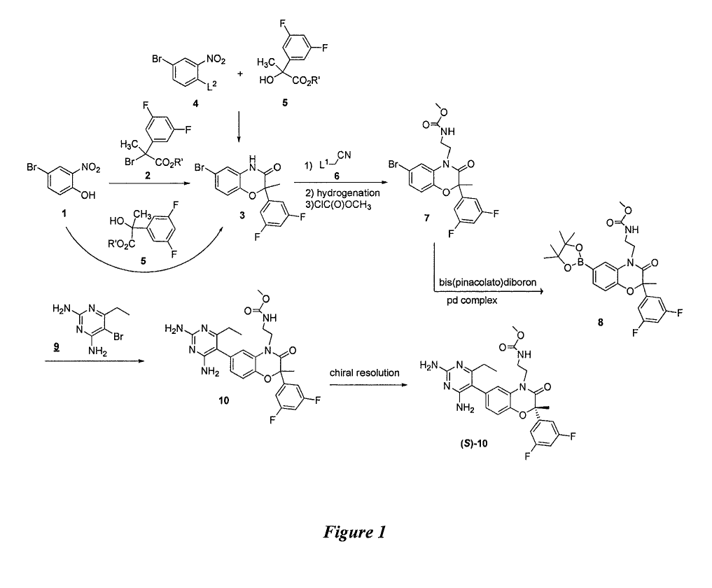

A variety of synthetic routes can be employed by the skilled artisan to prepare the compound and intermediates of the present invention. Schemes 1 and 2 (FIGS. 1-2) illustrate two approaches for the synthesis of certain substituted benzoxazinones. Related approaches are outlined in Powell, et al., BioOrg. & Med. Chem. 2007, 15, 5912-5949. The enantiomerically pure compound of formula (I) can be obtained using chiral separation techniques known in the art including chiral chromatographies, crystallizations and chiral resolution agents (see, Porter, et al. Pure & Appl. Chem., 1991, 63, 1119-1122; Beesely, et al. Chiral Chromatogrphy, 1st Ed. Wiley, 1999; Harold, J. Am. Chem. Soc. 1955, 77, 2910; and Yoshito, et al. Org. Process Res. Dev.; 2006; 10(5) pp 905-913). In Schemes 1 and 2 (FIGS. 1 and 2), L¹ is a leaving group, L² is a labile group that is capable of reacting with a nucleophile and R′ is a non-interfering substituent. During the reaction, protecting groups may be used. Examples of protecting groups can be found in T. W. Greene and P. G. Wuts, Protective Groups in Organic Chemistry, (Wiley, 4th ed. 2006), Beaucage and Iyer, Tetrahedron 48:2223-2311 (1992), and Harrison and Harrison et al., Compendium of Synthetic Organic Methods, Vols. 1-8 (John Wiley and Sons. 1971-1996). Representative amino protecting groups include formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC) and the like.

The reaction schemes (FIGS. 1 and 2), as noted above, provide certain synthetic routes that can be followed to access enantiomerically pure substituted (S)-benzoxazinones, particularly a compound of formula I. One of skill in the art will appreciate that modifications and refinements of the routes presented below are also within the scope of the present invention.

Scheme 1 (FIG. 1) shows three synthetic approaches to substituted (S)-benzoxazinones. All the approaches produce the common intermediate 3. In the first approach, compound 1, 4-bromo-2-nitrophenol is reacted with a substituted 2-bromo carboxylic ester, for example, alkyl-2-bromo-2-(3,5-difluorophenyl)propanoate 2 in the presence of a base, such as K₂CO₃, followed by reduction of the nitro group and in-situ cyclization to yield 1,4-benzoxazin-3-ones 3. In the second approach, the intermediate compound 3 can be synthesized by reacting compound 4 bearing a nitro group and a labile group L² with alkyl-2-(3,5-difluorophenyl)-2-hydroxypropanoate 5 through a nucleophilic aromatic substitution reaction in the presence of a base, such as NaH, followed by reduction of the nitro group and in-situ cyclization. The labile group L² can be a halide such as F. In the third approach, the intermediate compound 3 can be prepared by reacting compound 1 with a substituted 2-hydroxy carboxylic ester 5, such as alkyl-2-(3,5-difluorophenyl)-2-hydroxypropanoate under a Mitsunobu coupling condition, followed by reduction of the nitro group and in-situ cyclization. As shown in Scheme 1, substituted oxazinones 7 can be synthesized by nucleophilic substitution reaction of the intermediate compound 3 with compound 6 in the presence of a base, such as NaH, followed by selective hydrogenation of the cyano group and further reaction with methyl chloroformate. The bromo group of compound 7 can be readily converted to the boryl group of compound 8 by reacting compound 7 with bis(pinacolato)boron in the presence of a palladium complex under a Miyaura borylation condition. Compound 10 can be obtained by Suzuki coupling reaction of 5-bromo-2,4-diamino-6-ethyl-pyrimidine 9 with compound 8, for example, in the presence of a palladium complex. The (5)-enantiomer of compound (S)-10 having formula (I) can be obtained using chiral resolution techniques known in the art. Again, the choice of appropriate reaction conditions can be readily established by those of skill in the art.

Scheme 2 (FIG. 2) shows an alternative synthetic approach to enantiomerically pure substituted (S)-benzoxazinone compound of formula (I). This approach uses optically pure (S)-enantiomers of compounds (S)-2 and (S)-5 to react with compounds 1 and 4 to prepare (S)-enantiomer of intermediate (S)-3. Intermediate (S)-3 can be subsequently converted to enantiomerically pure compound (S)-10 using the synthetic sequence outlined in Scheme 2 (FIG. 2).

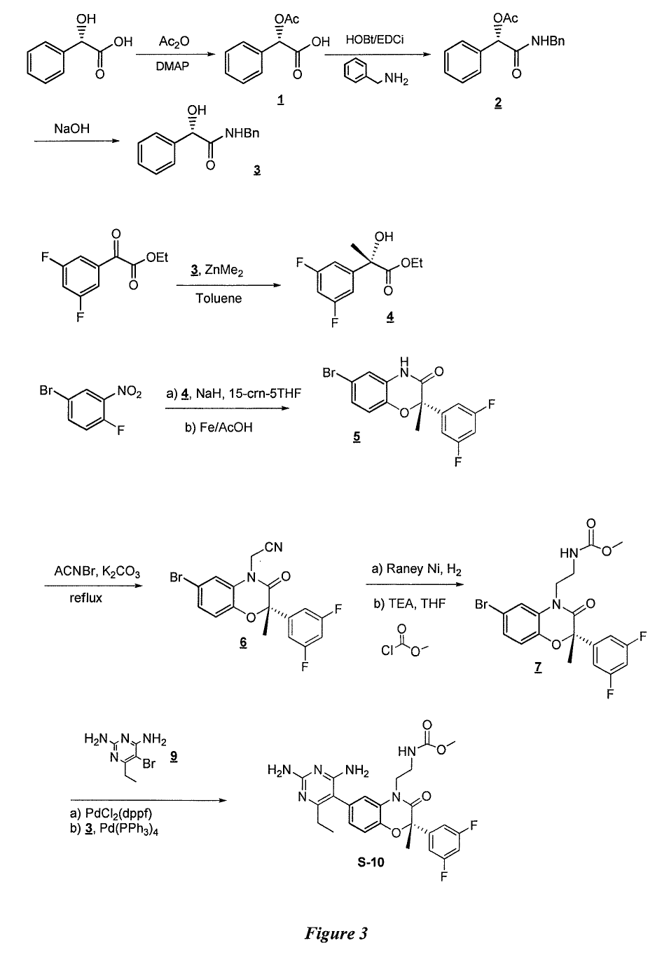

Scheme 3 (FIG. 3) shows another synthetic approach to enantiomerically pure substituted (S)-benzoxazinone compound of formula (I).

In another aspect, the present invention provides a method for preparing a compound of formula (I) and key intermediates as set forth in synthetic Schemes 1-3 (FIGS. 1-3). The exemplary key intermediates includes compounds 3, 7, 8 and 10 in FIG. 1; compounds (S)-3, (S)-7 and (S)-8 in FIG. 2; and compounds 4, 5, 6 and 7 in FIG. 3. The method for preparing a key intermediate includes contacting a precursor compound with a suitable agent under conditions sufficient to form the key intermediate. The precursor compound and the suitable agent are set forth in Schemes 1-3 (FIGS. 1-3). In one embodiment, the invention provides a method for preparing intermediate 5. The method includes contacting 4-bromo-2-nitro-1-fluoro benzene with compound 4 under conditions sufficient to form intermediate 5. In another embodiment, the invention provides a method for preparing a compound of formula (I). The method includes contacting compound 9 with intermediate 8, (S)-8 or 7 under conditions sufficient to form a compound of formula (I).

### IV. PHARMACEUTICAL COMPOSITIONS

In accordance with the present invention, a therapeutically effective amount of a compound of Formula (I) can be used for the preparation of a pharmaceutical composition useful for treating and/or preventing hypertension, congestive heart failure, stroke and myocardial infarction.

The compositions of the invention can include compounds of Formula (I), pharmaceutically acceptable salts thereof, a hydrate thereof or a hydrolysable precursor thereof. In general, the compound is mixed with suitable carriers or excipient(s) in a therapeutically effective amount. By a “therapeutically effective dose”, “therapeutically effective amount” or, interchangeably, “pharmacologically acceptable dose” or “pharmacologically acceptable amount”, it is meant that a sufficient amount of the compound of the present invention and a pharmaceutically acceptable carrier, will be present in order to achieve a desired result, e.g., alleviating a symptom or complication of hypertension.

The compounds of Formula (I) that are used in the methods of the present invention can be incorporated into a variety of formulations for therapeutic administration. More particularly, the compounds of Formula (I) can be formulated into pharmaceutical compositions by combination with appropriate, pharmaceutically acceptable carriers or diluents and can be formulated into preparations in solid, semi-solid, liquid or gaseous forms, such as tablets, capsules, pills, powders, granules, dragees, suspensions, gels, slurries, ointments, solutions, suppositories, injections, inhalants and aerosols. As such, administration of the compounds can be achieved in various ways, including oral, buccal, rectal, parenteral, intraperitoneal, intradermal, transdermal, intratracheal administration. Moreover, the compound can be administered in a local manner, in a depot or sustained release formulation. In addition, the compounds can be administered in a liposome.

The compounds of Formula (I) can be formulated with common excipients, diluents or carriers and compressed into tablets or formulated as elixirs or solutions for convenient oral administration or administered by intramuscular or intravenous routes. The compounds can be administered transdermally and can be formulated as sustained release dosage forms and the like. Compounds of Formula (I) can be administered alone, in combination with each other or they can be used in combination with other known compounds.

Suitable formulations for use in the present invention are found in Remington: The Science and Practice of Pharmacy, 21st ed., Lippincott Williams & Wilkins, Philadelphia, Pa., 2005, which is incorporated herein by reference. Moreover, for a brief review of methods for drug delivery, see, Langer, Science (1990) 249:1527-1533, which is incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

The pharmaceutical compositions for the administration of the compounds of this invention may conveniently be presented in unit dosage form and may be prepared by any of the methods well known in the art of pharmacy and drug delivery. All methods include the step of bringing the active ingredient into association with the carrier, which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the process or condition of diseases.

The pharmaceutical compositions containing compound of formula I may be in a form suitable for oral use, for example, as tablets, troches, lozenges, aqueous or oily suspensions, dispersible powders or granules, emulsions and self emulsifications as described in U.S. Patent Application 2002-0012680, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Oral solutions can be prepared in combination with, for example, cyclodextrin, PEG and surfactants.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols. Additionally, the compounds can be administered via ocular delivery by means of solutions or ointments. Still further, transdermal delivery of the subject compounds can be accomplished by means of iontophoretic patches and the like. For topical use, creams, ointments, jellies, solutions or suspensions, etc., containing the compounds of the present invention are employed. As used herein, topical application is also meant to include the use of mouth washes and gargles.

Dragee cores are provided with suitable coatings. For this purpose, concentrated sugar solutions can be used, which can optionally contain gum arabic, talc, polyvinyl pyrrolidone, carbopol gel, polyethylene glycol and/or titanium dioxide, lacquer solutions and suitable organic solvents or solvent mixtures. Dyestuffs or pigments can be added to the tablets or dragee coatings for identification or to characterize different combinations of active compound doses.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Alternatively, other deliveries for hydrophobic pharmaceutical compounds can be employed. Liposomes and emulsions are well known examples of delivery vehicles or carriers for hydrophobic drugs. In a presently preferred embodiment, long-circulating, i. e., stealth liposomes can be employed. Such liposomes are generally described in Woodle, et al., U.S. Pat. No. 5,013,556. The compounds of the present invention can also be administered by controlled release means and/or delivery devices such as those described in U.S. Pat. Nos. 3,845,770; 3,916,899; 3,536,809; 3,598,123; and 4,008,719.

The compounds of this invention may also be coupled with a carrier that is a suitable polymer as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the compounds of the invention may be coupled to a carrier that is a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like. In one embodiment of the invention, the compound of the invention is coupled to a polymer or semipermeable polymer matrix that is formed as a stent or stent-graft device.

Certain organic solvents such as dimethylsulfoxide (DMSO) also can be employed, although usually at the cost of greater toxicity. Additionally, the compounds can be delivered using a sustained-release, such as semipermeable matrices of solid hydrophobic polymers containing the therapeutic agent. Various types of sustained-release materials have been established and are well known by those skilled in the art. Sustained-release capsules can, depending on their chemical nature, release the compounds for a few hours up to over 100 days.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin and polymers such as polyethylene glycols.

Pharmaceutical compositions suitable for use in the present invention include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein.

For any compound used in the method of the present invention, a therapeutically effective dose can be estimated initially from cell culture assays or animal models.

Moreover, toxicity and therapeutic efficacy of the compounds described herein can be determined by standard pharmaceutical procedures in cell cultures or experimental animals, e.g., by determining the LD₅₀, (the dose lethal to 50% of the population) and the ED₅₀ (the dose therapeutically effective in 50% of the population). The dose ratio between toxic and therapeutic effect is the therapeutic index and can be expressed as the ratio between LD₅₀ and ED₅₀. Compounds which exhibit high therapeutic indices are preferred. The data obtained from these cell culture assays and animal studies can be used in formulating a dosage range that is not toxic for use in human. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g., Fingl et al. 1975 In: The Pharmacological Basis of Therapeutics, Ch. 1).

The dose administered to a patient, in the context of the present invention should be sufficient to effect a beneficial therapeutic response in the patient over time. The size of the dose also will be determined by the existence, nature, and extent of any adverse side-effects that accompany the administration of a particular compound in a particular patient. It will be understood, however, that the specific dose level for any particular patient will depend upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, or via a patch.

### V. SUSTAINED PR EXTENDED RELEASE FORMULATIONS

In one embodiment, a compound of the present invention is prepared for delivery in a sustained-release, controlled release, extended-release, timed-release or delayed-release formulation, for example, in semipermeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

The sustained or extended-release formulations can also be prepared using natural ingredients, such as minerals, including titanium dioxide, silicon dioxide, zinc oxide, and clay (see, U.S. Pat. No. 6,638,521, herein incorporated by reference). Exemplified extended release formulations that can be used in delivering a compound of the present invention include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Extended release formulations of particular interest include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817 and 5,296,483, each of which is hereby incorporated herein by reference. Those skilled in the art will readily recognize other applicable sustained release formulations.

The pharmaceutical formulations of the present invention can involve an extended release formulation, where the extended release formulation includes a compound of formula (I) and a pharmaceutically acceptable excipient, and the formulation exhibits an in vitro dissolution profile in simulated intestinal fluid medium comprising less than about 70% compound of formula (I) release after 1 hour, at least about 20% compound of formula (I) release after 4 hours, and at least about 30% compound of formula (I) release after 6 hours. In this embodiment, the extended release formulation exhibits an in vitro dissolution profile in simulated gastric fluid/simulated intestinal fluid (1 hour switchover) medium comprising less than about 80% compound of formula (I) release after 1 hour, at least about 30% compound of formula (I) release after 4 hours, and at least about 40% compound of formula (I) release after 6 hours.

In another embodiment, the formulation of the present invention contain an extended release component, where the extended release component includes a compound of formula (I) and a pharmaceutically acceptable excipient, and the extended release coating associated with extended release component exhibits an in vitro dissolution profile in simulated gastric fluid/simulated intestinal fluid (2 hour switchover) medium comprising less than about 10% compound of formula (I) release after 2 hours, at least about 40% compound of formula (I) release after 3 hours, and at least about 70% compound of formula (I) release after 6 hours. Preferably, the extended release coating associated extended release component exhibits an in vitro dissolution profile in simulated gastric fluid/simulated intestinal fluid (2 hour switchover) medium comprising less than about 10% compound of formula (I) release after 2 hours, at least about 50% compound of formula (I) release after 3 hours, and at least about 80% compound of formula (I) release after 6 hours. Most preferably, the extended release coating associated extended release component exhibits an in vitro dissolution profile in simulated gastric fluid/simulated intestinal fluid (2 hour switchover) medium comprising less than about 10% compound of formula (I) release after 2 hours, at least about 60% compound of formula (I) release after 3 hours, and at least about 90% compound of formula (I) release after 6 hours.

Appropriate in vitro dissolution testing methods for the formulation of the present invention are known to those of skill in the art. The USP paddle method refers to the Paddle and Basket Method as described in United States Pharmacopoeia, Edition XXII (1990). In particular, the USP paddle method of 50 rpm or 75 rpm in 900 ml simulated gastric fluid (SGF) (pH 1.2) or simulated intestinal fluid (SIF) (pH 6.8) at 37° C. may be used to determine the in vitro dissolution profiles according to the present invention.

The pharmaceutical formulation of the present invention are adapted to allow prolonged absorption of the active agent, which allows less frequent administration as compared to existing immediate-release formulations. As used herein, “prolonged absorption” means that the active agent is absorbed in vivo, under fasting conditions, over an extended period of time. In particular, the time period over which the majority (i.e., 80-90%) of the absorption occurs extends to about 7 or 8 hours after administration of the dosage form. Specifically, the median time period at which at least 80% of the active agent is absorbed from the dosage forms of the present invention is greater than 2.5 hours after administration, typically three to 4.5 hours after administration. By comparison, the median time period at which at least 80% of the active agent is absorbed from existing immediate-release formulations is 1.5 to two hours after administration. The period over which an active agent is absorbed from a dosage form can be calculated by deconvolution, using mathematical methods known to those of skill in the art.

The formulations of the present invention will exhibit an in vivo plasma profile comprising mean maximum compound of formula (I) levels from about 30 minutes to about 7 hours, often from about 2.5 hours to about 5.5 hours, after administration of a single dose to a fasting patient. At steady-state, the pharmaceutical dosage forms of the present invention will reach a minimum plasma concentration comparable to that obtained at steady-state from an immediate-release dosage form at a later time point, which will allow less frequent dosing. In particular, a 40 mg dosage form of the present invention, when administered twice daily, will deliver a mean steady-state area under the plasma concentration-time curve, a maximum plasma concentration, and a minimum plasma concentration similar to that of an immediate-release tablet formulation administered three times daily.

The formulations (preferably a tablet or capsule, which may contain beads, granules, particles, or a mixture thereof) may contain compound of formula (I) in the amount of from about 1 mg to about 200 mg (preferably from about 2.5 mg to about 100 mg) and can be used in the treatment of medical conditions including hypertension.

Among other formulations apparent to the skilled artisan, the solid oral formulation according to the present invention may be a tablet formulation, or a discrete unit-filled capsule formulation, or a sachet formulation. The discrete units of the present invention include beads, granules, pellets, spheroids, particles, tablets, pills, etc.

In one embodiment, the present invention provides an extended release formulation. The formulation includes a core comprising a compound of formula (I) or a pharmaceutically acceptable salt thereof, a pharmaceutically acceptable excipient and an extended release component; and an extended release coating associated with the core to provide for sustained release of compound of formula (I). The extended release component can comprise one or more ingredients which in combination with the extended release coating control the release of the active pharmaceutical ingredient, especially compound of formula (I), from the core in order to achieve a desired dissolution profile. The extended release component can comprise various water-insoluble materials. Examples of such water insoluble materials include hydrophobic polymers including, but are not limited to, one or more of ethyl cellulose, butyl cellulose, cellulose acetate, cellulose propionate, polyvinyl acetate, polyvinyl butyrate, ethyl acrylate-methyl methacrylate-ethyl ammonium trimethyl chloride methacrylate copolymer and N-vinyl-2-pyrrolidonecellulose ether, cellulose ester, polyvinyl ester, acrylic acid type polymer having a quaternary ammonium-alkyl group, and Plasdone® K-90, Povidone, homopolymer of N-vinyl-2-pyrrolidone. Preferably, the extended release agent comprises one or more of ethyl cellulose, butyl cellulose, cellulose acetate, cellulose propionate, polyvinyl acetate, polyvinyl butyrate, ethyl acrylate-methyl methacrylate-ethyl ammonium trimethyl chloride methacrylate copolymer and N-vinyl-2-pyrrolidone. Most preferably, the extended release agent comprises ethyl cellulose, such as Ethocel N-10 which is obtainable from Dow Chemical, Midland, Mich.

The extended-release component can also include a combination of hydrophilic and hydrophobic polymers. In this embodiment, once administered, the hydrophilic polymer dissolves away to weaken the structure of the extended-release component, and the hydrophobic polymer retards the water penetration and helps to maintain the shape of the drug delivery system.

The hydrophobic material may be selected from the group consisting of alkylcellulose, acrylic and methacrylic acid polymers and copolymers, shellac, zein, hydrogenated castor oil, hydrogenated vegetable oil, or mixtures thereof. In certain preferred embodiments, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid anhydride), polymethacrylate, polyacrylamide, poly(methacrylic acid anhydride), and glycidyl methacrylate copolymers. In alternate embodiments, the hydrophobic material is selected from materials such as one or more hydroxyalkyl celluloses such as hydroxypropyl methylcellulose. The hydroxyalkyl cellulose is preferably a hydroxy (C₁ to C₆) alkyl cellulose, such as hydroxypropylcellulose, hydroxypropylmethylcellulose, or preferably hydroxyethylcellulose. The amount of the hydroxyalkyl cellulose in the present oral dosage form is determined, inter alia, by the precise rate of active agents desired and may vary from about 1% to about 80%.

The core can be prepared in any manner, such as by utilizing any conventional manufacturing technique. Thus, the core can be produced by combining the active pharmaceutical ingredient and at least one agent capable of restricting release of the active ingredient with each other, and other ingredients. For example, the core may contain a wide variety of excipients, such as those ingredients that are conventionally included in the core of tablets, including diluents, glidents, binders, granulating solvent, granulating agents, anti-aggregating agents, buffers, lubricants.

For example, diluents can include, but are not limited to, one or more of sugars such as sucrose, lactose, mannitol, glucose; starch; microcrystalline cellulose; sorbitol, maltodextrin, whey, calcium phosphate; calcium sulfate; sodium sulfate or similar anhydrous sulfate; calcium lactate; lactose anhydrous; and lactose monohydrate. Preferably, the diluents comprise calcium sulfate and/or lactose monohydrate. Preferably, lactose anhydrous is not included in the core, because of its ability to absorb water from solvents, such as ethanol, thereby contributing to undesirable weight gain.

Examples of binders include, but are not limited to, one or more of polyvinyl alcohol, polyacrylic acid, polymethacrylic acid, polyvinyl pyrrolidone, sucrose, sorbitol, hydroxyethyl cellulose, hydroxypropylmethyl cellulose, hydroxypropyl cellulose, polyethylene glycols, gum arabic, gelatin, agar, starch, etc.

Lubricants and anti-aggregating agents include, but are not limited to, one or more of talc, magnesium stearate, calcium stearate, colloidal silica, stearic acid, waxes, hydrogenated vegetable oil, polyethylene glycols, sodium benzoate, sodium laurylsulfate, magnesium laurylsulfate and dl-leucine. Preferably, the lubricants include stearic acid and/or magnesium stearate.

Solvents include, but are not limited to, one or more of acetone, ethanol, isopropyl alcohol and/or methylene chloride. Preferably, the solvent for the core comprises ethanol. The use of chlorinated solvents, such as methylene chloride is preferably avoided. Therefore, the formulations preferably do not contain a chlorinated solvent, such as methylene chloride. The amount of solvent can be adjusted to provide a sufficient amount to achieve the formation of granules while not being of such a high amount as to turn the mixture into a large mass instead of granular in nature. Moreover, it is preferred to control the solvent addition time to effect adequate granulation. It is preferred that the temperature of granulating solution is preferably not be more than 26° C. at the time of addition to avoid lumping.

The ingredients can be combined and/or granulated in one or more procedures utilizing any appropriate techniques, such as by granulating in a low shear granulator, fluidized bed granulation, high shear granulator.

Following production of the granules for the core, the granules are dried under sufficient conditions to provide granules preferably having not more than 0.5 wt % alcohol, and preferably not more than 0.5 wt % water. Therefore, for commercial batches, it is preferred that the batches be dried at a preferred temperature of at about 50° C., preferably for at least 16 hours to ensure solvent removal, such as ethanol and/or water.

The cores are compressed into tablets to a hardness of preferably between about 2 and 6 kP, with a preferred value being about 4 kP.

In one embodiment, the present invention provides extended release particles. The extended release particles slowly release compound of formula (I) when ingested and exposed to gastric fluids, and then to intestinal fluids. The extended release profile of the formulations of the invention can be altered, for example, by increasing or decreasing the thickness of the retardant coating, i.e., by varying the amount of overcoating. The resultant solid extended release particles may thereafter be placed in a gelatin capsule in an amount sufficient to provide an effective extended release dose when ingested and contacted by an environmental fluid, e.g., gastric fluid, intestinal fluid or dissolution media. The particles may be overcoated with an aqueous dispersion of a hydrophobic or hydrophilic material to modify the release profile. The aqueous dispersion of hydrophobic material preferably further includes an effective amount of plasticizer, e.g. triethyl citrate. Preformulated aqueous dispersions of ethylcellulose, such as Aquacoat® or Surelease®, may be used. If Surelease® is used, it is not necessary to separately add a plasticizer.

The release of the therapeutically active agent from the extended release formulation of the present invention can be further influenced, i.e., adjusted to a desired rate, by the addition of one or more release-modifying agents. The release-modifying agent may be organic or inorganic and include materials that can be dissolved, extracted, or leached from the coating in the environment of use. The pore-formers may comprise one or more hydrophilic materials such as hydroxypropyl methylcellulose. The release-modifying agent may also comprise a semi-permeable polymer. In certain preferred embodiments, the release-modifying agent is selected from hydroxypropyl methylcellulose, lactose, metal stearates, and mixtures thereof.

The extended release coating is deposited in a location that is external to the core, and is preferably layered directly onto the core. The extended release coating is formulated to provide in conjunction with the extended release component in the core a desired dissolution profile.

In one embodiment, the extended release coating comprises a film preferably a combination of hydrophobic and hydrophilic polymers. Preferably, the active pharmaceutical ingredient is not present in the extended release coating and is only present in the core.

The hydrophobic polymer constituting the film is not particularly limited, provided that it has film forming ability, and is insoluble in water, but soluble in a water-miscible organic solvent. Examples of hydrophobic polymers include, but are not limited to, one or more of ethyl cellulose, butyl cellulose, cellulose acetate, cellulose propionate, polyvinyl acetate, polyvinyl butyrate, ethyl acrylate-methyl methacrylate-ethyl ammonium trimethyl chloride methacrylate copolymer and N-vinyl-2-pyrrolidonecellulose ether, cellulose ester, polyvinyl ester, acrylic acid type polymer having a quaternary ammonium-alkyl group, and Plasdone® K-90, Povidone, homopolymer of N-vinyl-2-pyrrolidone. Preferably, there may be included, for example, ethyl cellulose, butyl cellulose, cellulose acetate, cellulose propionate, polyvinyl acetate, polyvinyl butyrate, ethyl acrylate-methyl methacrylate-ethyl ammonium trimethyl chloride methacrylate copolymer and N-vinyl-2-pyrrolidone. Most preferably, the hydrophobic polymeric substance comprises ethyl cellulose, such as Ethocel N-10, which is obtainable from Dow Chemical, Midland, Mich.

The hydrophilic polymer may be a water-soluble polymeric substance, including, but are not limited to, one or more of methyl cellulose, polysaccharides optionally having a sulfuric acid group such as pullulan, dextrin, alkali metal alginate, etc.; polysaccharides having a hydroxy-alkyl group or a carboxyalkyl group such as hydroxypropyl cellulose, hydroxypropylmethyl cellulose, carboxymethyl cellulose sodium, etc.; methyl cellulose, polyvinyl pyrrolidone, polyvinyl alcohol or polyethylene glycol, etc. may be included. Preferably, the hydrophilic polymeric substance comprises methyl cellulose, such as Methocel E-15, which is obtainable from Dow Chemical, Midland, Mich.

Changing the weight ratio of hydrophobic polymer to hydrophilic polymer in the extended release coating affects the dissolution of the compound. Therefore, the weight ratio of hydrophobic polymer to hydrophilic polymer can be varied for each specific formulation to obtain desired dissolution profiles for the formulation. Preferable weight ratios for the hydrophobic polymer to hydrophilic polymer include weight ratios of 55-65:45-35, with one preferred weight ratio of hydrophobic polymer to hydrophilic polymer being 57:43.

The solvent for the extended release coating can comprise various solvents including, but are not limited to, acetone, ethanol, isopropyl alcohol (IPA) and/or methylene chloride. Preferably, the solvent for the extended release coating comprises ethanol. The use of chlorinated solvents, such as methylene chloride is preferably avoided. Various combinations of solvents can be used, such as, but not limited to, ethanol and isopropyl alcohol, and combinations of isopropyl alcohol:water, denatured alcohol:water and ethyl alcohol:water.

Coating of the extended release coating can be performed using various techniques, such as conventional coating techniques such as perforated pan, or using fluidized bed technique. These methods are commonly understood by individuals who are practicing the art tablet manufacture. The angle of spray guns utilized in spayed on the coating can be varied, such as, but not limited to between 30.degree. and 75.degree., with one preferred angle being 65.degree.

In certain embodiments, the coating comprises an aqueous dispersion of a hydrophobic polymer, the inclusion of an effective amount of a plasticizer in the aqueous dispersion of hydrophobic polymer can further improve the physical properties of the film. For example, because ethylcellulose has a relatively high glass transition temperature and does not form flexible films under normal coating conditions, it is necessary to plasticize the ethylcellulose before using it as a coating material. Generally, the amount of plasticizer included in a coating solution is based on the concentration of the film-former, e.g., most often from about 1 percent to about 50 percent by weight of the film-former. Concentration of the plasticizer, however, is preferably determined after careful experimentation with the particular coating solution and method of application.

Examples of suitable plasticizers for ethylcellulose include water-insoluble plasticizers such as dibutyl sebacate, diethyl phthalate, triethyl citrate, tributyl citrate, and triacetin, although other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to, citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is an especially preferred plasticizer for aqueous dispersions of ethyl cellulose. It has further been found that addition of a small amount of talc reduces the tendency of the aqueous dispersion to stick during processing and acts a polishing agent.

One commercially available aqueous dispersion of ethylcellulose is Aquacoat® which is prepared by dissolving the ethylcellulose in a water-immiscible organic solvent and then emulsifying the ethylcellulose in water in the presence of a surfactant and a stabilizer. After homogenization to generate submicron droplets, the organic solvent is evaporated under vacuum to form a pseudolatex. The plasticizer is not incorporated into the pseudolatex during the manufacturing phase. Thus, prior to using the pseudolatex as a coating, the Aquacoat® is mixed with a suitable plasticizer.

Another aqueous dispersion of ethylcellulose is commercially available as Surelease® (Colorcon, Inc., West Point, Pa., USA). This product is prepared by incorporating plasticizer into the dispersion during the manufacturing process. A hot melt of a polymer, plasticizer (dibutyl sebacate), and stabilizer (oleic acid) is prepared as a homogeneous mixture which is then diluted with an alkaline solution to obtain an aqueous dispersion which can be applied directly onto substrates.

In one preferred embodiment, the acrylic coating is an acrylic resin lacquer used in the form of an aqueous dispersion, such as that which is commercially available from Rohm Pharma under the trade name Eudragit®. In additional preferred embodiments, the acrylic coating comprises a mixture of two acrylic resin lacquers commercially available from Rohm Pharma under the trade names Eudragit® RL 30 D and Eudragit® RS 30 D. Eudragit® RL 30 D and Eudragit® RS 30 D are copolymers of acrylic and methacrylic esters with a low content of quaternary ammonium groups, the molar ratio of ammonium groups to the remaining neutral (meth)acrylic esters being 1:20 in Eudragit® RL 30 D and 1:40 in Eudragit® RS 30 D. The mean molecular weight is about 150,000 Daltons. The code designations RL (high permeability) and RS (low permeability) refer to the permeability properties of these agents. Eudragit® RL/RS mixtures are insoluble in water and in digestive fluids, however, coatings formed from them are swellable and permeable in aqueous solutions and digestive fluids.

The Eudragit® RL/RS dispersions may be mixed together in any desired ratio in order to ultimately obtain a extended release formulation having a desirable dissolution profile. Desirable extended release formulations may be obtained, for instance, from a retardant coating derived from one of a variety of coating combinations, such as 100% Eudragit® RL; 50% Eudragit® RL and 50% Eudragit® RS; or 10% Eudragit® RL and 90% Eudragit® RS. One skilled in the art should recognize that other acrylic polymers may also be used, for example, Eudragit® L. In addition to modifying the dissolution profile by altering the relative amounts of different acrylic resin lacquers, the dissolution profile of the ultimate product may also be modified, for example, by increasing or decreasing the thickness of the retardant coating.

In preferred embodiments of the present invention, the stabilized product is obtained by subjecting the coated substrate to oven curing at a temperature above the T_{g} of the plasticized acrylic polymer for the required time period, the optimum values for temperature and time for the particular formulation being determined experimentally. In certain embodiments of the present invention, the stabilized product is obtained via an oven curing conducted at a temperature of about 45° C. for a time period from about 1 to about 48 hours. It is also contemplated that certain products coated with the extended release coating of the present invention may require a curing time longer than 24 to 48 hours, e.g., from about 48 to about 60 hours or more.

The coating solutions preferably contain, in addition to the film-former, plasticizer, and solvent system (i.e., water), a colorant to provide elegance and product distinction. Color may be added to the solution of the therapeutically active agent instead of, or in addition to the aqueous dispersion of hydrophobic material. For example, color may be added to Aquacoat® via the use of alcohol or propylene glycol based color dispersions, milled aluminum lakes and opacifiers such as titanium dioxide by adding color with shear to the water soluble polymer solution and then using low shear to the plasticized Aquacoat®. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retardant effect of the coating.

Spheroids or beads coated-with the therapeutically active agents can be prepared, for example, by dissolving the therapeutically active agents in water and then spraying the solution onto a substrate, for example, non pareil 18/20 beads, using a Wuster insert. Optionally, additional ingredients are also added prior to coating the beads in order to assist the binding of the active agents to the beads, and/or to color the solution, etc. For example, a product that includes hydroxypropyl methylcellulose with or without colorant (e.g., Opadry®, commercially available from Colorcon, Inc.) may be added to the solution and the solution mixed (e.g., for about I hour) prior to application onto the beads. The resultant coated substrate, beads in this example, may then be optionally overcoated with a barrier agent to separate the therapeutically active agent from the hydrophobic extended release coating. An example of a suitable barrier agent is one that comprises hydroxypropylmethylcellulose. However, any film-former known in the art may be used. It is preferred that the barrier agent does not affect the dissolution rate of the final product.

In one embodiment, the extended release coating, delayed or delayed-sustained release coating is an enteric coating. All commercially available pH-sensitive polymers may be used to form the enteric coating. The drug coated with the enteric coating is minimally or not released in the acidic stomach environment of approximately below pH 4.5. The drug should become available when the enteric layer dissolves at the higher pH present in the intestine; after a suitable delayed time; or after the unit passes through the stomach. The preferred duration of drug release time is in the range of up to 7 hours after dosing under fasting conditions.

Enteric polymers include cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl esters such as, for instance, materials known under the trade name Eudragit® L12.5, Eudragit® L100, or Eudragit® S12.5, S100 (Röhm GmbH, Darmstadt, Germany) or similar compounds used to obtain enteric coatings. Aqueous colloidal polymer dispersions or re-dispersions can be also applied, e.g., Eudragit® L 30D-55, Eudragit®V L100-55, Eudragit® S100, Eudragit® preparation 4110D c; Aquateric®, Aquacoat® CPD 30 (FMC Corp.); Kollicoat MAE® 30D and Kollicoat MAE® 30DP (BASF); Eastacryl® 30D (Eastman Chemical, Kingsport, Tenn.).

The enteric polymers used in this invention can be modified by mixing with other known coating products that are not pH sensitive. Examples of such coating products include the neutral methacrylic acid esters with a small portion of trimethylammonioethyl methacrylate chloride, sold currently under the trade names E Eudragit®, Eudragit® RL, Eudragit® RS; a neutral ester dispersion without any functional groups, sold under the trade names Eudragit® NE30D and Eudragit® NE30; and other pH independent coating products.

The enteric coating will substantially envelop the extended-release component. The term “substantially envelop” is intended to define the total or near-total enclosure of a component. Such an enclosure includes, preferably, at least about 80% enclosure, more preferably at least about 90% enclosure, and even more preferably at least about 99% enclosure.

In one embodiment, the dosage form is a capsule formulation, which capsule contains a combination of beads containing compound of formula (I) in an immediate-release formulation and beads containing compound of formula (I) in an enteric-coated extended-release formulation. In this preferred embodiment, the enteric-coated extended-release beads contain two layers that control the rate of release of compound of formula (I).

The extended-release beads are prepared by coating the compound of formula (I) on sugar spheres, then adding a first polymer layer, followed by a second, pH-sensitive enteric polymer layer. Preferably, the outer, enteric coating will dissolve at pH of about 5.5 or greater. The inner layer may contain a pH-sensitive or pH-independent polymer as described herein, provided that the inner layer functions to provide sustained release of the compound of formula (I) upon dissolution of the outer enteric coat. The inner extended-release polymer layer will be applied in an amount such that, in combination with the outer, enteric-coating, the enteric-coated controlled-release component yields the in vitro dissolution profile described herein. In a particularly preferred embodiment, the inner layer comprises an enteric polymer that dissolves at about pH 6.0.

An embodiment of the present invention provides for a free flowing formulation comprising compound of formula (I). The term “free flowing” as used herein, means formulations that pass through a patient's digestive system without impediment or mechanism to slow passage. Thus, for example, the term “free flowing” would exclude gastric raft type formulations, which are designed to reside in the stomach for extended periods as in, e.g., U.S. Pat. No. 5,651,985.

### VI. EXAMPLES

The following abbreviations are used in the Examples and throughout the description of the invention.
DMAP: 4-Dimethylaminopyridine
DMF: Dimethyl formamide
DCM: Dichloromethane
EtOAc: Ethyl acetate
EDC: 1-ethyl-3-(3-dimethylaminopropyl)carbodiimide
HOBt: N-Hydroxybenzotriazole
THF: Tetrahydrofuran
TEA: Triethylamine
dppf: 1,1′-Bis(diphenylphosphino)ferrocene
SHR: Spontaneously hypertensive rat
IP: Intraperitoneal
BP: Blood Pressure

### Example 1

### Preparation of (S)-2-acetoxy-2-phenylacetic acid (1)

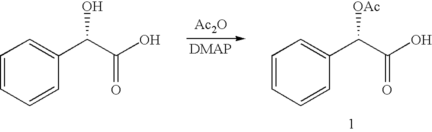

To a round bottom flask charged with the Mandelic acid (15 g, 111 mmol) was added 100 mL of acetic anhydride. After 5 min stirring, 10 mL of anhydrous ether was added and followed by a catalytic amount of DMAP. The resultant reaction mixture was kept at r t for additional 16 h. The reaction mixture was evaporated under reduced pressure to give a amorphous material which in turn diluted with EtOAc (50 mL). The solution was washed with 1N HCl (2×10 mL), water (2×10 mL) and followed by brine solution (10 mL). The org. layer was then dried over MgSO₄ and filtered. The resultant solution was concentrated under reduced pressure. The desired product was isolated by a column chromatography using 20% EtOAc in hexane (15.8 g, 81 mmol). Yield 73%; R_{f} 0.54 (1:1 hex:EtOAc) ¹H NMR (CDCl₃, δ) 11.5 (s, 1H), 7.43 (m, 5H), 5.99 (s, 1H), 2.20 (s, 3H).

### Example 2

### Preparation of (S)-(benzylcarbamoyl)(phenyl)methyl acetate (2)

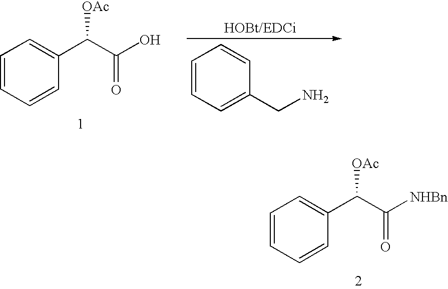

To a DMF solution (7 mL) containing the (S)-2-acetoxy-2-phenylacetic acid (1, 41 mmol) was added EDC and HOBt (2.5 equiv each) and the reaction mixture was stirred for 5 min at ambient temperature prior to the addition of benzyl amine (1.5 equiv). The reaction mixture was then stirred at room temperature for 16 h. The reaction mixture was diluted with EtOAc (25 mL), which was washed with 1N HCl (10 mL×2), sat. NaHCO₃ (10 mL×2), water (10 mL) and followed by brine. The org. layer was then dried over MgSO₄, and filtered. The resultant product solution was concentrated under reduced pressure to give a pale yellow liquid. The crude product was then purified by a column chromatography using a 30% (v/v) EtOAc in hexane as a mobile phase. Yield: 73%; ¹H NMR (CDCl₃, δ) 7.21 (m, 10H), 6.45 (br. s, 1H), 5.08 (s, 1H), 4.43 (q, 2H), 3.64 (s, 1H).

### Example 3

### Preparation of (S)-N-benzyl-2-hydroxy-2-phenylacetamide (3)

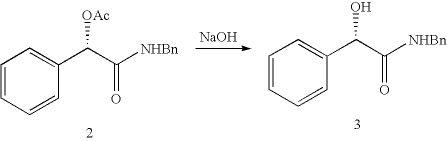

To a MeOH solution (10 mL) containing 0.5N NaOH/MeOH solution (7 mL) was added (S)-(benzyl-carbamoyl)(phenyl)methyl acetate (2, 3.0 g, 10.6 mmol). The reaction was complete less than 1 h at room temperature. The reaction mixture was evaporated under reduced pressure to yield a pale yellow liquid which was re-dissolved in EtOAc (100 mL). The solution was then washed with 1N HCl (15 mL), saturated NaHCO₃ (15 mL) and water. Once washed with brine (5 mL), the organic layer was separated, dried over MgSO₄ and filtered. The filtrate was concentrated under reduced pressure to give a white powder (7.73 mmol). Yield: 73%; ¹H NMR (CDCl₃, δ) 7.23 (m, 10H), 6.45 (br. s, 1H), 5.08 (s, 1H), 4.43 (q, 2H), 3.64 (s, 1H).

### Example 4

### Preparation of (S)-Ethyl 2-(3,5-difluorophenyl)-2-hydroxypropanoate (4)

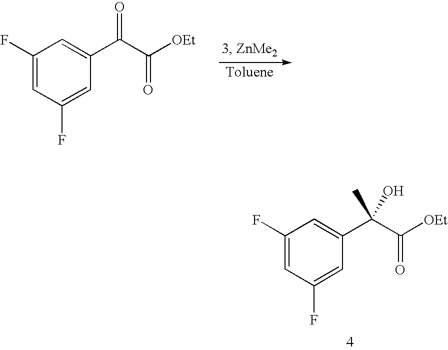

To a toluene solution (2 mL) containing the mandelamide (3, 1.4 g, 5.81 mmol) was added the commercially available substrate, ethyl 2-(3,5-difluorophenyl)-2-oxoacetate and followed by dimethylzinc (8.7 mL of 2M solution in Toluene, 3 equiv, 17.4 mmol) at 0° C. The reaction mixture was stirred at 0° C. for 4 h. After the reaction was quenched with 5 mL of HCl and the same amount of water, EtOAc was added to the mixture. The organic layer was drawn off to a separatory funnel where it was washed with saturated NaHCO₃ (2×10 mL), water (2×15 mL) and brine. The organic layer was dried over MgSO₄ and filtered. The filtrate was then concentrated under reduced pressure to give an off-white solid. The white solid was washed with n-hexane where starting material and the desired product was dissolved. The catalyst was filtered off. The remaining hexane solution was concentrated under reduced pressure to give a pale yellow liquid. Yield 58%; ¹H NMR (CDCl₃, δ) 7.13 (m, 2H), 6.73 (m, 1H), 4.26 (q, 2H), 3.93 (s, 1H), 1.74 (s, 3H), 1.29 (t, 3H).

### Example 5

### Preparation of (S)-7-bromo-3-(3,5-difluorophenyl)-3-methyl-benzo[2,3-b][1,4]oxazin-2(3H)-one (5)

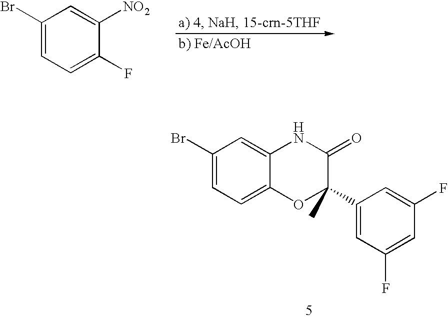

Step a) A solution of 1.00 g (4.35 mmol) of (S)-2-(3,5-difluorophenyl)-2-hydroxy-propionicacid ethyl ester in 20 mL of anhydrous THF was treated with 325 mg (8.13 mmol) of a 60% NaH dispersion in mineral oil. The resulting grey suspension was stirred at room temperature for 10 min. Then, 5 drops of 15-crown-5 THF were added via syringe, followed by 1.49 g (6.78 mmol) of 4-bromo-2-nitro-1-fluoro benzene. The resultant orange solution was stirred at room temperature for 17 h. Excess hydride was quenched by the careful addition of MeOH. The solution was diluted with EtOAc, and then washed with brine, dried over MgSO4, filtered, and concentrated to dryness. A flash column chromatography was used to isolate the desired intermediate (hex 100% to 10% EtOAc/Hex). The intermediate was used for the next step without further purification.

Step b) Step 2. The residue was dissolved in 20 mL of glacial acetic acid, treated with 3.79 g (67.8 mmol) of iron dust, and heated in a 60° C. oil bath for 4 h. The resulting gray suspension was cooled to room temperature, diluted with EtOAc, and filtered through a Celite pad. The filtrate was concentrated to dryness. The solid residue was dissolved in EtOAc and washed with H₂O (3×) and saturated aqueous NaHCO₃ . The organic layer was dried over MgSO₄, which was filtered and concentrated. The desired product was obtained via purification by a flash-column chromatography (SiO₂, 100% hexanes then gradient to 5% EtOAc/hexanes) gave 1.72 g of 5 as a clear oil (80%, 2 steps). ¹H NMR (CD₃OD, δ) 9.53 (s, 1H), 7.26 (dd, 1H), 7.05 (d, 4H), 6.62 (m, 1H), 1.93 (s, 3H); MS(ESI+): m/z 354.0, 356.9 [M+1], Br isotopes, 1:1 ratio; MS(ESI+).

### Example 6

### Preparation of 2-((S)-7-bromo-3-(3,5-difluorophenyl)-2,3-dihydro-3-methyl-2-oxobenzo[2,3-b][1,4]oxazin-1-yl)acetonitrile (6)

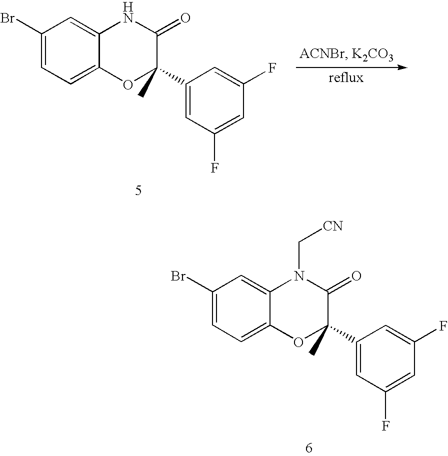

A solution of 5 (2.09 g, 5.89 mmol) in 30 mL of anhydrous acetonitrile was treated with 1.722 mL (11.8 mmol) of bromoacetonitrile and 2.0 g (14.78 mol) of K₂CO₃, and heated at reflux for 16 h. The resulting suspension was cooled to room temperature, diluted with EtOAc, and filtered through a Celite plug. The filtrate was washed with brine, dried over MgSO₄, and concentrated under reduced pressure. Purification by a flash column chromatography (SiO₂, 5% EtOAc/hexanes then gradient to 20% EtOAc/hexanes) gave 2.11 g as amorphous foam. Yield 91%; ¹H NMR (CDCl₃, δ ppm) 7.32 (m, 1H), 7.04 (m, 4H), 6.88 (m, 1H), 6.70 (m, 1H), 4.85 (dd, 2H), 1.93 (s, 3H); ¹³C NMR (CDCl₃, δ ppm) 164.9, 164.8, 161.6, 149.6, 143.9, 142.3, 124.7, 123.2, 114.3, 113.1, 108.5, 108.1, 104.9, 104.6, 104.3, 82.6; MS(ESI+): m/z 394.0, 396 [M+1], Br isotopes, 1:1 ratio.

### Example 7

### Preparation of methyl 2-((S)-7-bromo-3-(3,5-difluorophenyl)-2,3-dihydro-3-methyl-2-oxobenz[2,3-b][1,4]oxazin-1-yl)ethylcarbamate (7) (or (S)-7 in FIG. 2)

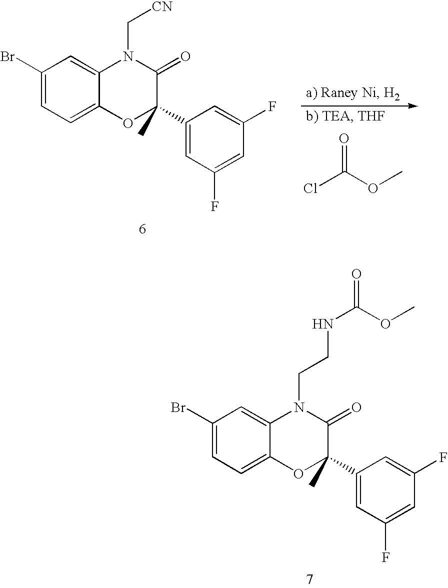

Methyl Cyanopyridoxazinone (6, 0.97 g) in 5 mL of dry THF was placed in a container (250 mL), which was charged with Raney Ni that was washed several times with dry THF (ca. 1 g in 10 mL THF). The reaction mixture was then supplied with H₂ gas in a balloon, and stirred at room temperature. Periodically, to the mixture was added methyl chloroformate and TEA. The progress of the reaction was monitored by TLC and addition of methyl chloroformate and TEA. The total reaction time was 24 h. The reaction mixture was filtered through a pad of Celite®, and the collected liquid was diluted with EtOAc (20 mL). The combined org. layer was concentrated under reduced pressure to give yellow amorphous foam. The desired product was isolated with a column chromatography (a gradient of 10% EtOAc to 25% in hex) 1.29 g. Yield 67%; ¹H NMR (CDCl₃, δ ppm) 8.13 (s, 1H), 7.60 (s, 1H), 6.95 (d, 2H), 6.73 (m, 1H), 4.15 (m, 1H), 3.90 (m, 1H), 3.65 (s, 3H), 3.40 (d, 2H), 1.93 (s, 3H); ¹³C NMR (CDCl₃, δ ppm) 165.4, 164.9, 161.6, 157.3, 143.4, 142.7, 125.2, 124.9, 123.2, 114.3, 108.5, 108.4, 108.1, 104.5, 104.2, 103.8, 82.6, 52.1, 41.4, 38.4, 37.4; MS(ESI+): m/z 455, 456 [M+1], Br isotopes, 1:1 ratio.

### Example 8

### Preparation of Methyl 2-((S)-7-(2,4-diamino-6-ethylpyrimidin-5-yl)-3-(3,5-difluorophenyl)-2,3-dihydro-3-methyl-2-oxobenz[2,3-b][1,4]oxazin-1-yl)ethylcarbamate ((S)-10)

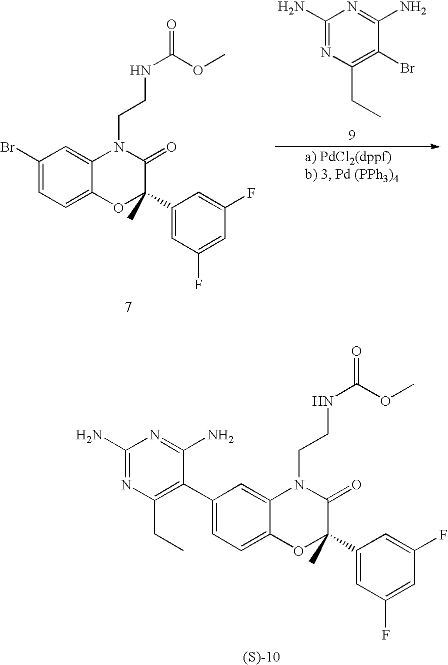

Step a) A solution of 7 (0.40 g, 0.877 mmol), bis(pinacolato)diboron (0.267 g, 1.05 mmol) and KOAc (0.258 g 2.63 mmol) in 10 mL of anhydrous 1,4-dioxane was degassed by applying a reduced pressure for 2 min while agitated. A PdCl₂(dppf)-CH₂Cl₂ complex (60 mg, 0.156 mmol) was added, and the resulting orange red suspension was heated in a 95° C. oil bath for 22 h. After cooling to room temperature, the black mixture was diluted with EtOAc, filtered through Celite, and concentrated.

Step b) The residue was dissolved in a mixture of 1,4-dioxane (10 mL) and H₂O (1.5 mL). To the solution, were added CsOH H₂O (0.445 g, 2.63 mmol 3 equiv), LiCl (0.119, 2.63 mmol, 3 equiv) and 5-bromo-6-ethylpyrimidine-2,4-diamine (2, 0.236 g, 1.10 mmol, 1.25 equiv). The resultant suspension was degassed by applying intermittent vacuum until no bubbling was observed. Pd(PPh₃)₄ (200 mg, 0.173 mmol) was then added to the reaction mixture, which was then heated to 100° C. for 18 h. The mixture was diluted with EtOAc (50 mL), dried over MgSO₄, filtered through a pad of Celite and the filtrate was concentrated to give a dark-brown oil. The desired product (S-10) was obtained by a flash column chromatography using a gradient of 0 to 10% MeOH in DCM (120 mg). Yield 23%; ¹H NMR (CDCl₃, δ ppm) 7.28 (m, 1H), 6.90-7.17 (m, 4H), 6.83-6.89 (m, 1H), 4.25 (m, 2H), 3.60 (s, 3H), 3.55 (m, 2H), 2.37 2.20 (2m, 2H rotamer), 2.05 (s, 3H), 1.17 1.00 (2m, 3H rotamer); MS(ESI+): m/z 513 [M+1].

### Example 9

### Biological Procedure and Results

### General Information

Cardiovascular data is collected from non-restrained animals implanted with radiotelemetry devices.

### Equipment

Software: DSI Ponemah Physiology Platform (version 4.8), Dataquest OpenART Gold DSI telemetry implants, DSI receiver plates and other hardware.

### Procedures

1. SHR rats, 7-10 weeks of age weighing 150-300 g, are implanted with radiotelemetry devices. Transmitters are placed subcutaneously in the ventral abdominal region and blood pressure is monitored from the femoral artery. Animals will be allowed to recoverat least 10 days prior to data collection.

2. Signal quality is verified by placing individual animals into a plastic solid-bottom cage set on top of a receiver plate.

3. Telemeters are turned on using a magnet. An AM radio is utilized to determine whether signals are being generated by the unit. Telemetry implants are turned off when data is not being collected in order to conserve battery power.

4. The software is set up according to the pertinent instruction manual.

5. At least 1 hour of baseline is collected from undisturbed animals prior to each dosing. Following test article administration, approximately 24 hours of data is collected from undisturbed animals to assess hemodynamic effects.

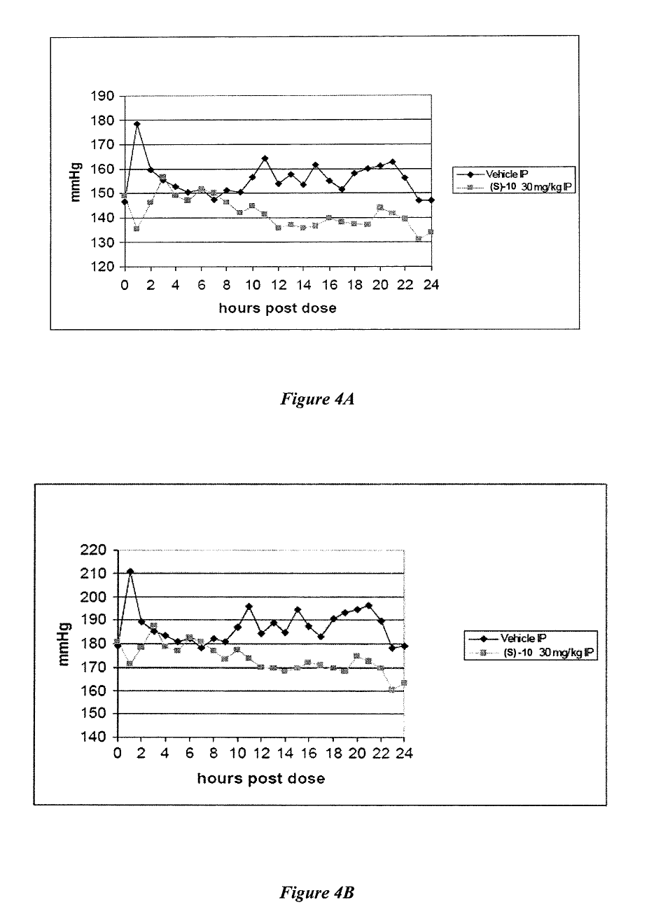

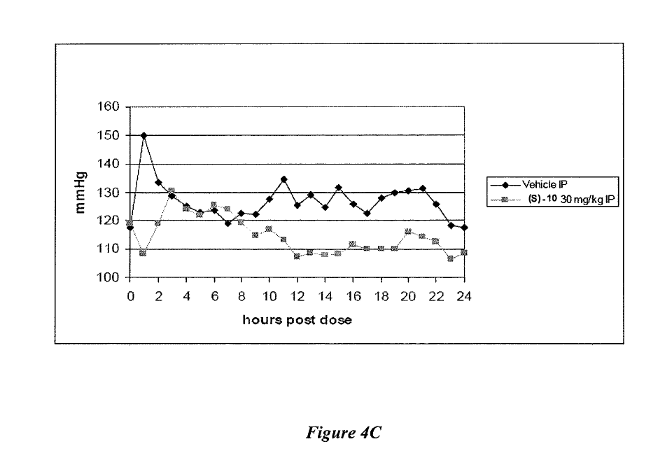

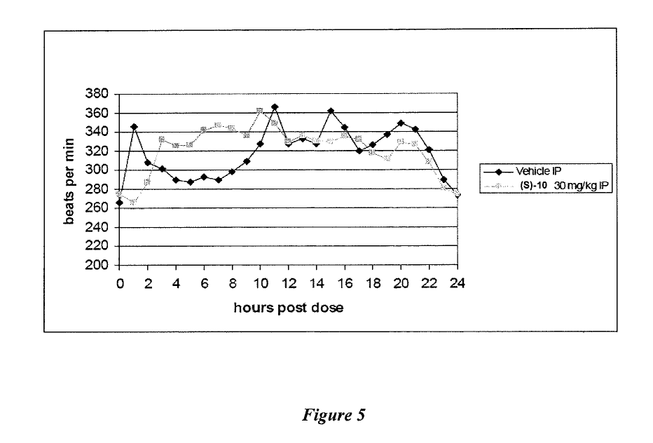

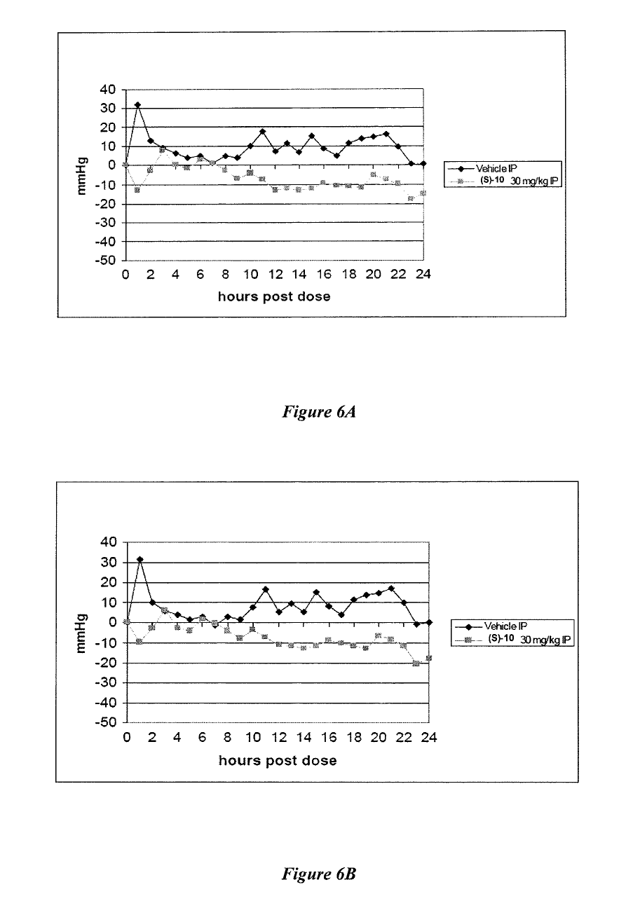

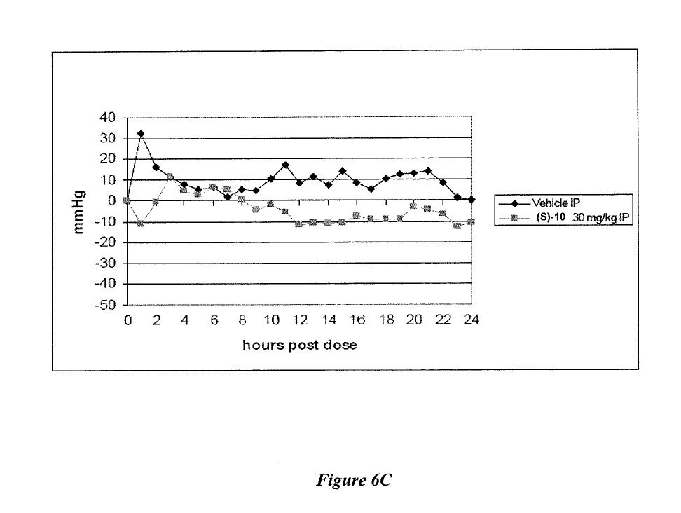

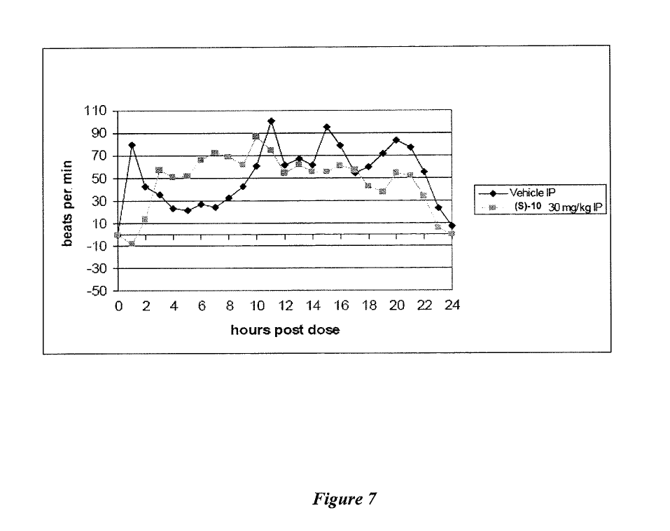

Comparisons of BP and heart rates of SHR rats after administration of 30 mg/kg IP with vehicle IP over a 24 hours time period are shown in FIGS. 4-7.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A compound having formula (I): 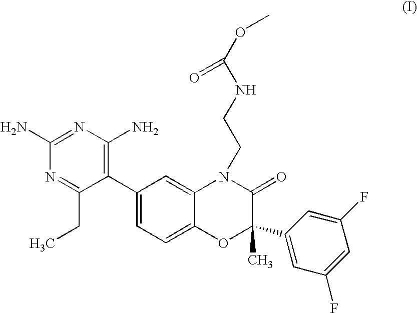
and a pharmaceutically acceptable salt or solvate thereof, wherein the stereochemical configuration at the carbon atom bearing —CH₃ and 3,5-difluorophenyl is (S).

2. A pharmaceutical composition comprising a pharmaceutically acceptable excipient and a compound of claim 1.

3. An extended release formulation comprising:
a core comprising a compound of claim 1, a pharmaceutically acceptable excipient and an extended release component; and
an extended release coating associated with the core to provide for sustained release of compound of formula (I).

4. The formulation of claim 3, wherein upon oral administration of a pharmaceutical dosage form of the formulation, the median time period at which at least 80% of compound of formula (I) is absorbed, in vivo, under fasting conditions, is greater than 2.5 hours.

5. The formulation of claim 3, wherein upon oral administration of a pharmaceutical dosage form of the formulation, the median time period at which at least 80% of compound of formula (I) is absorbed, in vivo, under fasting conditions, is from about 3 hours to about 4.5 hours.

6. The formulation of claim 3, wherein extended release coating comprises a first, inner polymer layer and a second, outer polymer layer.

7. The formulation of claim 6, wherein said second, outer polymer layer comprises a pH-sensitive polymer.

8. The formulation of claim 3, wherein the first, inner polymer layer comprises a pH-sensitive or pH-independent polymer.

9. The formulation of claim 3, wherein the extended release component comprises a hydrophobic polymer.

10. The formulation of claim 9, wherein the hydrophobic polymer is selected from the group consisting of cellulose acetate phthalate, cellulose acetate trimellitate, hydroxypropyl methylcellulose phthalate, polyvinyl acetate phthalate, carboxymethylethylcellulose, co-polymerized methacrylic acid, methacrylic acid methyl esters, and mixtures thereof.

11. The formulation of claim 3, wherein the extended release coating comprises hydrophobic polymer and hydrophilic polymer.

12. The formulation of claim 3, wherein the compound of formula (I) is in an amount of about 1 mg to about 200 mg.

13. The formulation of claim 3, wherein the formulation has a pharmaceutical dosage form of a tablet.

14. The formulation of claim 3, wherein the formulation has a pharmaceutical dosage form of a capsule.
